(19) United States
(12) Patent Application Publication
(10) Pub. No.: US 2003/0149413 A1
(43) Pub. Date: Aug. 7, 2003

(54) Superabsorbent composite and absorbent articles including the same

(76) Inventor: Fouad D. Mehawej, Forest Lake, MN (US)

Correspondence Address: Julie Post, H.B. Fuller Company, 1200 Willow Lake Blvd., P.O. Box 64683, St. Paul, MN, 55164-0683, US

(21) Appl. No.: 10/066,935

(22) Filed: Feb. 4, 2002

(51) Int. Cl. A61F 13/15

(52) U.S. Cl. 604/368; 604/378

(57) ABSTRACT

An absorbent article having a core that includes a composite including superabsorbent polymer and a high loft nonwoven web impregnated with the superabsorbent polymer, the superabsorbent polymer having been formed in situ and being present in the composite in an amount from 10% by weight to about 90% by weight.

### BACKGROUND

The invention relates to superabsorbent composites.

Absorbent articles such as disposable diapers and feminine hygiene products often include various layers made from fibrous nonwoven webs and a core of compressed cellulose fibers, often referred to as “fluff” or “pulp,” held together with chemical binder, or through physical entanglement and compression.

The fibrous nonwoven webs of disposable diaper constructions are often positioned as a top sheet and an acquisition layer. These nonwoven webs are made from synthetic polymers, tend to be have little to no absorbent capacity and, in the case of the top sheet and the acquisition layer, function to disperse liquid to enable it to transfer to a greater area of a second layer, e.g., the core, and to maintain a dry feel on the wearer's skin.

The absorbent core is designed to absorb and hold liquid. Many efforts have been made to increase the absorbent capacity and rate of absorption of cellulose fiber cores. Superabsorbent polymers in particulate and powder form have been added to disposable diaper and feminine napkin cores to improve the absorbent capacity and rate of absorption of the articles. In the case of a diaper construction, for example, superabsorbent powder or particulate is sifted in with the absorbent core material during the diaper manufacturing process. Superabsorbent particles are very fine and tend to become airborne during processing. Superabsorbent particles also generally do not adhere to the substrate and tend to migrate and shift during manufacturing, shipping, handling, use or a combination thereof. The movement of the superabsorbent particles can lead to insufficient liquid storage capacity in some areas and excess liquid storage capacity in other areas of the article.

Cellulose fiber cores have disadvantages in that they have weak integrity in both, dry and wet, conditions. Additional compression and embossing processes designed to improve the integrity of cellulose fiber cores often result in a stiffer core having a poor absorption rate. In addition, during the manufacture of cellulose fiber cores loose fibers become air-borne and may present a safety hazard.

Airlaid or pre-made absorbent cores provide a thinner core product and eliminate problems related to the processing of loose cellulose fibers, but they tend to lack integrity. Chemical binders are often used to improve the integrity of airlaid cores. However, chemical binders tend to negatively impact the absorption rate and absorption capacity of the core.

### SUMMARY

In one aspect, the invention features a disposable diaper having a core that includes a composite including superabsorbent polymer (i.e., a polymer that is capable of absorbing many times its weight of water) and a high loft nonwoven web impregnated with the superabsorbent polymer, the superabsorbent polymer having been formed in situ, the composite including from 10% by weight to about 90% by weight superabsorbent polymer. In one embodiment, the composite includes at least 50% by weight superabsorbent polymer. In another embodiment, the composite includes at least 60% by weight superabsorbent polymer. In other embodiments, the composite includes at least 70% by weight superabsorbent polymer. In one embodiment, the composite includes at least 80% by weight superabsorbent polymer.

In some embodiments, the nonwoven web has a basis weight of greater than 22 g/m². In other embodiments, the nonwoven web has a basis weight from about 25 g/m² to less than 300 g/m². In another embodiment, the nonwoven web has a basis weight of at least 55 g/m². In one embodiment, the nonwoven web has a basis weight of at least 90 g/m². In some embodiments, the nonwoven web has a basis weight of at least 100 g/m².

In other embodiments, the nonwoven web has a density less than 0.01 g/cm³. In another embodiments, the nonwoven web has a density less than 0.008 g/cm³. In some embodiments, the nonwoven web has a density from about 0.002 g/cm³ to about 0.009 g/cm³. In other embodiments, the nonwoven web has a density from about 0.007 g/cm³ to about 0.009 g/cm³.

In one embodiment, the composite exhibits a saline absorption capacity under a load of 0.3 psi of at least 10 g 0.9% saline/g composite. In some embodiments, the composite exhibits a saline absorption capacity under a load of 0.3 psi of at least 15 g 0.9% saline/g composite. In other embodiments, the composite exhibits a saline absorption capacity under a 0.3 psi load of at least 20 g 0.9% saline/g composite.

In another embodiment, the composite exhibits a water absorption capacity of at least 20 g water/g composite. In some embodiments, the composite exhibits a water absorption capacity of at least 30 g water/g composite. In other embodiments, the composite exhibits a water absorption capacity of at least 40 g water/g composite.

In another embodiment, the composite exhibits a dry tensile strength of at least 2000 g/25.4 mm. In some embodiments, the composite exhibits a dry tensile strength of at least 2500 g/25.4 mm. In one embodiment, the composite exhibits a wet tensile strength of at least 150 g/25.4 mm. In other embodiments, the composite exhibits a wet tensile strength of at least 400 g/25.4 mm. In some embodiments, the composite exhibits a wet tensile strength of at least 450 g/25.4 mm.

In other embodiments, the disposable diaper further includes a top sheet, an acquisition layer, a cellulose fiber layer, an impermeable layer or a combination thereof. In one embodiment, the core further includes cellulose fibers and the disposable diaper further includes an acquisition layer, the cellulose fibers being disposed between the acquisition layer and the composite. In another embodiment, the disposable diaper further includes an acquisition layer and an impermeable layer, the core being disposed between the acquisition layer and the impermeable layer. In some embodiments, the disposable diaper further includes a second nonwoven web and an acquisition layer, the acquisition layer being disposed between the core and the second nonwoven web.

In some embodiments, the superabsorbent polymer includes the reaction product of a polymer derived from an α-β-ethylenically unsaturated carboxylic acid monomer, the polymer including neutralized carboxylic acid groups, and a crosslinking agent. In another embodiment, the α-β-ethylenically unsaturated carboxylic acid is selected from the group consisting of methacrylic acid, crotonic acid, maleic acid, maleic acid anhydride, itaconic acid, fumaric acid, and mixtures thereof. In one embodiment, the polymer includes polyacrylic acid.

In other embodiments, the superabsorbent polymer remains disposed within the matrix of the high loft web when contacted with an aqueous composition.

In another embodiment, the core further includes cellulose fibers, the composite being disposed in regions on the cellulose fibers.

In one embodiment, the core includes a plurality of strips of the composite.

In another aspect, the invention features an absorbent article having a core that includes a composite including superabsorbent polymer, and a high loft nonwoven web impregnated with the superabsorbent polymer, the superabsorbent polymer having been formed in situ, the composite including from 10% by weight to about 90% by weight superabsorbent polymer. In one embodiment the article is a feminine napkin, incontinence pad or a mattress pad.

In other aspects, the invention features an absorbent article having a core that includes a composite including superabsorbent polymer, and a nonwoven web impregnated with said superabsorbent polymer, the nonwoven web having loft and a density of no greater than 0.025 g/m³, the superabsorbent polymer having been formed in situ, the composite including from 10% by weight to about 90% by weight superabsorbent polymer. In one embodiment, the nonwoven web has a density no greater than 0.023 g/m³.

In other aspects, the invention features a method of making an absorbent article that includes impregnating a high loft nonwoven web with an aqueous composition including a superabsorbent polymer precursor and a crosslinking agent, drying the composition to form a composite including from 10% by weight to about 90% by weight superabsorbent polymer, and incorporating the composite in an absorbent article.

The invention features a disposable diaper that includes a superabsorbent core having a high concentration of superabsorbent polymer and exhibiting good liquid absorption capacity, good liquid absorption capacity under load and a good rate of liquid absorption. The core exhibits improved wet strength relative to the cellulose fiber cores of existing diapers.

The invention also features a disposable article that includes a superabsorbent core that is thin relative to existing cellulose fiber cores and can be used in place of or in addition to cellulose fiber cores. Absorbent articles constructed to include the core can be made to be comfortable and provide good wearability. The superabsorbent polymer remains fixed in place and does not migrate under dry conditions and remains within the matrix under wet conditions.

The invention also features a simple core manufacturing process.

Other features of the invention will be apparent from the following description of the preferred embodiments thereof, and from the claims.

### DETAILED DESCRIPTION

The absorbent core includes a superabsorbent composite that includes a superabsorbent polymer and a nonwoven web impregnated with the superabsorbent polymer, the superabsorbent polymer having been formed in situ, i.e., in place on the nonwoven web from an aqueous superabsorbent polymer composition. The superabsorbent polymer-impregnated web includes superabsorbent polymer throughout the three-dimensional matrix of the web. The superabsorbent polymer may reside on the fibers of the web and, optionally, in the interstices of the web.

The nonwoven web preferably is a high loft nonwoven web, i.e., a nonwoven web having a density of no greater than 0.01 gram per cubic centimeter (g/cm³). The three dimensional structure of a high loft nonwoven web matrix includes passageways, e.g., channels, through which liquid (e.g., water, blood, and urine) can migrate, e.g., wick. When liquid contacts the superabsorbent composite, the superabsorbent polymer begins to expand. The high loft nonwoven web and the fibers of the high loft nonwoven web preferably expand when contacted with liquid. The three-dimensional nature of the high loft matrix and the expansion of the web accommodate liquid present in the web, liquid traveling into the web, and the swelling superabsorbent polymer. The expansion of the web enables the superabsorbent composite to absorb a greater volume of liquid relative to a nonwoven web having a relatively high basis weight and high density, and being essentially two-dimensional.

The three-dimensional matrix of the high loft nonwoven web also assists in maintaining the swollen, i.e., gelled, superabsorbent polymer in the web matrix. Preferably the superabsorbent polymer gel does not migrate out of the high loft matrix and does not transfer or move during use of the absorbent article. At least one additional layer of nonwoven web can be placed between a user and the composite to prevent the gelled superabsorbent polymer from contacting the user.

The superabsorbent composite exhibits good saline absorption under load, high saline absorption capacity and high water absorption capacity. Preferably the superabsorbent composite exhibits a saline absorption capacity under load of at least 10 g 0.9% saline solution/g composite under a 0.3 pound per square inch (psi) load, more preferably at least 15 g 0.9% saline solution/g composite, most preferably at least 20 g 0.9% saline solution/g composite. The superabsorbent composite also preferably exhibits a water absorption capacity of at least 20 g water/g composite, more preferably at least 40 g water/g composite, most preferably at least 70 g water/g composite within a period of 10 minutes.

The superabsorbent composite exhibits good dry strength and maintains strength and integrity when wet. Preferably the superabsorbent composite exhibits a dry tensile strength of at least 2000 g/25.4 mm, more preferably at least 2500 g/25.4 mm, most preferably a dry tensile strength of at least 3000 g/25.4 mm, and a wet tensile strength of at least 150 g/25.4 mm, more preferably, at least 400 g/25.4 mm, most preferably at least 500 g/25.4 mm.

The superabsorbent composite preferably includes an amount of superabsorbent polymer sufficient to provide good absorption capacity while maintaining a web having a degree of softness and flexibility suitable for its intended use. As the concentration of superabsorbent polymer present in the composite increases, the softness and flexibility of the composite decreases. Useful superabsorbent composites include at least 10% by weight superabsorbent polymer, at least 50% by weight superabsorbent polymer, at least 60% by weight superabsorbent polymer and at least 90% by weight superabsorbent polymer. The composite preferably includes from about 10% by weight to about 70% weight superabsorbent polymer, more preferably from about 10% by weight to about 70% by weight superabsorbent polymer, most preferably from about 30% by weight to about 60% by weight superabsorbent polymer.

The superabsorbent polymer is applied to the high loft web in the form of an aqueous composition, which, upon drying, crosslinks to form the superabsorbent polymer. The aqueous composition can be dried according to various methods including, e.g., with air, heat or a combination thereof (e.g., by passing the composite through an oven).

The aqueous composition includes a superabsorbent polymer precursor (e.g., an alkali soluble polyelectrolyte) and a crosslinking agent. As the aqueous composition dries, the superabsorbent polymer precursor crosslinks to form the superabsorbent polymer. Particularly useful aqueous superabsorbent compositions include polymers of water soluble monomers including, e.g., at least partially neutralized polymers derived from α,β-ethylenically unsaturated mono- or dicarboxylic acid monomers and acid anhydride monomers, and a crosslinking agent. The polymers can be fully neutralized. The phrase “partially neutralized” refers to the presence of neutralized carboxylic acid groups in the polymer. Useful water soluble monomers include acrylic acid, methacrylic acid, crotonic acid, maleic acid, maleic anhydride, itaconic acid and fumaric acid. Any free radial generating source may be used to initiate polymerization of the monomers including, e.g., peroxides and persulfates. The polymerization of such monomers produces an alkali soluble polyelectrolyte. Useful aqueous superabsorbent compositions are described in PCT Patent Application No. WO 00/61642 (Anderson et al.) and incorporated herein. A useful commercially available aqueous superabsorbent polymer composition is available under the trade designation FULATEX PD-8081-H from H. B. Fuller Company (Vadnais Heights, Minn.).

Useful crosslinking agents include any substance that will react with the hydrophilic groups of the aqueous solution polymer. Useful crosslinking agents include, e.g., zirconium ions, ferric aluminum ions, chromic ions, titanium ions and combinations thereof, and aziridine. A variety of suitable crosslinking agents are described in U.S. Pat. No. 4,090,013 and incorporated herein. One example of a useful commercially available crosslinking agent is BACOTE 20 ammonium zirconyl carbonate available from Magnesium Elektron Inc. (Flemington, N.J.).

Other useful aqueous superabsorbent compositions include aqueous polymer compositions having a pH of from 4 to 6, which can be adjusted with metal hydroxide or alkaline earth metal hydroxide, where the aqueous polymer compositions includes α,β-ethylenically unsaturated carboxylic acid monomer and a softening monomer in an amount effective to yield a polymer having a Tg<140° C., and a crosslinking salt, e.g., zirconium crosslinking salt. Suitable superabsorbent polymers are described, e.g., in U.S. Pat. No. 5,693,707 (Cheng et al.) and incorporated herein.

The aqueous superabsorbent polymer composition can be applied to the high loft web using a variety of techniques including, e.g., soaking, spraying, printing, and coating, and can be present throughout the web or in discreet locations on the web. Preferably the web is impregnated with superabsorbent polymer such that it exists throughout the web matrix.

Useful high loft nonwoven webs have a basis weight of greater than 22 g/m² for a web thickness (i.e., caliper) of at least 1 millimeter (mm), preferably at least 30 g/m², more preferably at least 60 g/m², more preferably at least 80 g/cm², most preferably at least 100 g/cm². The high loft nonwoven web can vary in thickness depending on the application. Suitable high loft nonwoven webs have a thickness of at least 10 mm, more preferably at least 15 mm. The high loft nonwoven web also has a density no greater than 0.01 g/m³, preferably from about 0.002 g/cm³ to about 0.009 g/cm³, more preferably from about 0.007 g/cm³ to about 0.009 g/cm³. Other useful nonwoven webs with loft have a density of no greater than 0.025 g/m³, and no greater than 0.023 g/m³.

The nonwoven web includes synthetic polymer fibers of, e.g., polyester, polyolefin (e.g., polypropylene, polyethylene, and copolymers of polyolefins and polyesters), polyamide, polyurethane, polyacrylonitrile, and combinations thereof including copolymers thereof, bicomponent (e.g., sheath core) fibers and combinations thereof. Preferably the nonwoven web is resilient and includes resilient fibers (e.g., polyester fibers). The fibers are preferably curly and are mechanically and physically entangled.

Nonwoven webs can be formed using a variety of methods including, e.g., air-laying, wet laying, garneting and carding, and melt blown and spun bond techniques.

The superabsorbent composite is useful as the core or a component of the core of various absorbent articles (preferably a disposable absorbent article) including, e.g., disposable diapers, feminine hygiene products (e.g., sanitary napkins), bandages, wound care products, surgical pads, adult incontinence pads, and bibs. The superabsorbent composite can replace or compliment cores that include traditional materials such as cellulose fibers and other fluff materials. The superabsorbent composite can be present in the absorbent article in the form of a continuous web, positioned in regions on another component of the absorbent article and combinations thereof. The regions of composite can be positioned and can be in various configurations including e.g., randomly or in a pattern (e.g., strips), and combinations thereof. The composite can also be maintained in position within the article with an adhesive composition.

The absorbent article can optionally include other components including, e.g., a body fluid pervious top sheet, an acquisition layer, a second absorbent layer (e.g., a second core or fibrous layer), a body fluid impermeable back sheet, and combinations thereof. The acquisition layer preferably is capable of dispersing liquid to the surface of the core. The second absorbent layer may include loose fibers, fibers held together through a binder, compressed fibers and combinations thereof. The fibers of the second absorbent layer may be natural fibers (e.g., wood pulp, jute, cotton, silk and wool and combinations thereof), synthetic fibers including (e.g., nylon, rayon polyester, acrylics, polypropylenes, polyethylene, polyvinyl chloride, polyurethane, and combinations thereof), and combinations thereof. The superabsorbent composite can be disposed between any of the components and preferably is disposed between the body fluid pervious top sheet and a body fluid impermeable back sheet, more preferably between an acquisition layer and a body fluid impermeable back sheet.

The invention will now be described further by way of the following examples. All parts, ratios, percents and amounts stated in the Examples are by weight unless otherwise specified.

### EXAMPLES

Test Procedures

Test procedures used in the examples include the following.

Total Water Absorbency

The total water absorbency (g/g) is the weight of tap water in grams (g) that each gram of a 100 cm² sample of composite absorbs in 10 minutes. A 100 cm² (10 cm×10 cm) sample of dry composite is weighed (WD). The sample is then submerged in tap water for 10 minutes. The wet and swollen composite is placed on a pre-weighted metal screen (WS) for one minute. The excess water present in the sample is allowed to drain. The wet sample and the screen are then weighed (WW).

The total water absorbency (Twa) is calculated according to the following equation:

Twa=[(WW−WS)−WD]/WD

and reported in g absorbed water/g composite

Total 0.9% Saline Solution Absorbency Under Load

The total 0.9% saline absorbency (g/g) is the weight of 0.9% saline (g) that each gram of a 100 cm² sample of composite absorbs in 10 minutes. The total 0.9% saline absorbency is determined by weighing a 100 cm² (10 cm×10 cm) sample of dry composite (WD). The sample is placed in a receptacle and a metal mesh screen and brass weights are placed on top of the sample. Both the metal screen and the weights have the same size as (i.e., are coextensive with) the sample, and the total weight of the metal mesh screen and brass weights must exert 0.3 psi on the sample. A sufficient amount of 0.9% saline solution is poured into the receptacle to submerge the absorbent sample. After 10 minutes, the weight and metal screen are removed. The absorbent sample (WW) is then promptly weighed.

The total 0.9% saline absorbency under load (AUL) is calculated according to the following equation:

0.9% Saline AUL=(WW−WD)/WD

and reported in g absorbed 0.9% saline solution/g composite

Dry Tensile Strength

A 4 inch×1 inch strip of sample composite is cut and ½ inch strips of masking tape are wrapped at each of the 1 inch wide ends of the composite strip. The composite strip is then placed between the jaws of an Instron tester (Instron Corp., Canton, Mass.) and tensile strength is measured at a 12 inch/min cross-head speed. The average tensile strength of 5 samples is reported as the Dry Tensile Strength in g/in.

Wet Tensile Strength

A 4 inch×1 inch strip of sample composite is cut and ½ inch strips of masking tape are wrapped at each of the 1 inch wide ends of the composite strip. The composite strip is then soaked in water for 5 minutes, gently patted dry of excess water and then promptly tested by placing the sample between the jaws of an Instron tester. Tensile strength is measured at a 12 inch/min cross-head speed. The average tensile strength of 5 samples is reported as the Wet Tensile Strength in g/in.

% Superabsorbent Polymer (SAP) Loading

The percent superabsorbent polymer present in the composite is determined by weighing the web prior to treatment with superabsorbent polymer, weighing the dried composite after treatment with superabsorbent polymer, subtracting to find the weight of superabsorbent polymer in the composite, and dividing the weight of the superabsorbent polymer by the total weight of the composite.

The results are reported as % SAP.

Controls 1 and 2

Samples were prepared by saturating polyester fiber nonwoven webs having the properties set forth in Tables 1 and 2 with an aqueous superabsorbent polymer composition of 95 parts FULATEX PD-8081-H aqueous superabsorbent polymer (23% solids) (H. B. Fuller Company, Vadnais Heights, Minn.) and 5 parts BACOTE 20 ammonium zirconyl carbonate (40% active as supplied) (Magnesium Elektron Inc., Flemington, N.J.). The webs were dried and weighed to determine % superabsorbent polymer present in the composite.

### Examples 1-4

Superabsorbent composites were prepared by saturating polyester fiber nonwoven webs having the properties set forth in Table 1 with an aqueous superabsorbent polymer composition of 95 parts FULATEX PD-8081-H aqueous superabsorbent polymer (23% solids) and 5 parts BACOTE 20 ammonium zirconyl carbonate (40% active as supplied) (Magnesium Elektron Inc., Flemington, N.J.). The webs were dried and weighed to determine % superabsorbent polymer present in the composite.

The samples of Control 1 and Examples 1-4 were tested according to the above-described methods to determine wet and dry tensile strength. The weight and thickness of the samples were also determined. The results are reported in Table 1.

TABLE 1

| | | | | Dry | | Tensile | Tensile |
|---|---|---|---|---|---|---|---|
| | | | | Composite | Wet | Strength | Strength |
| | Basis | | | Basis | Composite | (Dry) | (Wet) |
| | Weight | Thickness | | Weight | Thickness | g/25.4 | g/25.4 |
| Sample | (g/m²) | (mm) | % SAP | (g/m²) | (mm) | mm | mm |
| Control 1 | 22 | 0.06 | 82 | 122 | 1.5 | 2820 | 480 |
| Example 1 | 30 | 2 | 83 | 176 | 2.3 | 2430 | 385 |
| Example 2 | 60 | 5 | 73 | 222 | 11 | 2480 | 410 |
| Example 3 | 60 | 5 | 90 | 600 | 13 | 2660 | 460 |
| Example 4 | 100 | 14 | 76 | 416 | 16 | 2870 | 406 |

### Examples 5-17

Superabsorbent composites were prepared according to Example 1 with the exception that the nonwoven webs had the basis weight and density set forth in Table 2 and the amount of superabsorbent polymer applied to the web was controlled to achieve a composite having the % superabsorbent polymer indicated in Table 2.

The samples of Controls 1 and 2 and Examples 5-17 were tested according to the above-described methods to determine the water absorbent capacity and 0.9% Saline absorbency under load (AUL). The results are reported in Table 2.

TABLE 2

| | | | Untreated Web | | SAP-Containing Composite | | |
|---|---|---|---|---|---|---|---|
| | | | | 0.9% Saline | | | 0.9% Saline |
| | | | | AUL | | | AUL |
| | | | Water | (g 0.9% | | Water | (g 0.9% |
| | Basis | | Absorbency | saline | | Absorbency | saline |
| | Weight | Density | (g water/g | solution/g | | (g water/g | solution/g |
| Sample | (g/m²) | (g/mm³) | composite) | composite) | % SAP | composite) | composite) |
| Control 1 | 22 | ND | 4 | 2 | 82 | 18 | 10 |
| Example 5 | 30 | 0.0227 | 6 | 4 | 83 | 24 | 12 |
| Example 6 | 30 | 0.0227 | 6 | 4 | 71 | 18 | 10 |
| Example 7 | 30 | 0.0227 | 6 | 4 | 57 | 12 | 14 |
| Example 8 | 30 | 0.0227 | 6 | 4 | 52 | 10 | 14 |
| Example 9 | 60 | 0.0024 | 10 | 5 | 87 | 31 | 12 |
| Example 10 | 60 | 0.0076 | 18 | 5 | 90 | 65 | 17 |
| Example 11 | 60 | 0.0076 | 18 | 5 | 79 | 46 | 15 |
| Example 12 | 60 | 0.0076 | 18 | 5 | 73 | 37 | 15 |
| Example 13 | 60 | 0.0076 | 18 | 5 | 62 | 31 | 13 |
| Example 14 | 60 | 0.0076 | 18 | 5 | 50 | 28 | 14 |
| Example 15 | 100 | 0.0083 | 20 | 7 | 76 | 34 | 18 |
| Example 16 | 100 | 0.0083 | 20 | 7 | 59 | 33 | 17 |
| Example 17 | 100 | 0.0083 | 20 | 7 | 51 | 31 | 21 |
| Control 2 | 300 | 0.046 | 30 | 15 | 50 | 22 | 12 |

ND = not determined

Other embodiments are within the claims. Although the superabsorbent composite has been described with respect to disposable article cores, the superabsorbent composite is also useful in various other absorbent article applications including, e.g., wipes, towels, facial tissue, mops, and agricultural applications (e.g., to maintain moisture). The composite can also be combined with at least one other nonwoven web in a layered construction.

What is claimed is:

1. A disposable diaper having a core that comprises a composite comprising:
superabsorbent polymer; and
a high loft nonwoven web impregnated with said superabsorbent polymer,
said superabsorbent polymer having been formed in situ,
said composite comprising from 10% by weight to about 90% by weight superabsorbent polymer.

2. The disposable diaper of claim 1, wherein said composite comprises at least 50% by weight superabsorbent polymer.

3. The disposable diaper of claim 1, wherein said composite comprises at least 60% by weight superabsorbent polymer.

4. The disposable diaper of claim 1, wherein said composite comprises at least 70% by weight superabsorbent polymer.

5. The disposable diaper of claim 1, wherein said composite comprises at least 80% by weight superabsorbent polymer.

6. The disposable diaper of claim 1, wherein said nonwoven web has a basis weight of greater than 22 g/m².

7. The disposable diaper of claim 1, wherein said nonwoven web has a basis weight from about 25 g/m² to less than 300 g/m².

8. The disposable diaper of claim 1, wherein said nonwoven web has a basis weight of at least 55 g/m².

9. The disposable diaper of claim 1, wherein said nonwoven web has a basis weight of at least 90 g/m².

10. The disposable diaper of claim 1, wherein said nonwoven web has a basis weight of at least 100 g/m².

11. The disposable diaper of claim 1, wherein said nonwoven web has a density less than 0.01 g/cm³.

12. The disposable diaper of claim 1, wherein said nonwoven web has a density less than 0.008 g/cm³.

13. The disposable diaper of claim 1, wherein said nonwoven web has a density from about 0.002 g/cm³ to about 0.009 g/cm³

14. The disposable diaper of claim 1, wherein said nonwoven web has a density from about 0.007 g/cm³ to about 0.009 g/cm³.

15. The disposable diaper of claim 1, wherein said composite exhibits a saline absorption capacity under a load of 0.3 psi of at least 10 g 0.9% saline/g composite.

16. The disposable diaper of claim 1, wherein said composite exhibits a saline absorption capacity under a load of 0.3 psi of at least 15 g 0.9% saline/g composite.

17. The disposable diaper of claim 1, wherein said composite exhibits a saline absorption capacity under a 0.3 psi load of at least 20 g 0.9% saline/g composite.

18. The disposable diaper of claim 1, wherein said composite exhibits a water absorption capacity of at least 20 g water/g composite.

19. The disposable diaper of claim 1, wherein said composite exhibits a water absorption capacity of at least 30 g water/g composite.

20. The disposable diaper of claim 1, wherein said composite exhibits a water absorption capacity of at least 40 g water/g composite.

21. The disposable diaper of claim 1, wherein said composite exhibits a dry tensile strength of at least 2000 g/25.4 mm.

22. The disposable diaper of claim 1, wherein said composite exhibits a dry tensile strength of at least 2500 g/25.4 mm.

23. The disposable diaper of claim 1, wherein said composite exhibits a wet tensile strength of at least 150 g/25.4 mm.

24. The disposable diaper of claim 1, wherein said composite exhibits a wet tensile strength of at least 400 g/25.4 mm.

25. The disposable diaper of claim 1, wherein said composite exhibits a wet tensile strength of at least 450 g/25.4 mm.

26. The disposable diaper of claim 1 further comprising a top sheet, an acquisition layer, a cellulose fiber layer, an impermeable layer or a combination thereof.

27. The disposable diaper of claim 1, wherein said core further comprises cellulose fibers, said disposable diaper further comprising an acquisition layer, said cellulose fibers being disposed between said acquisition layer and said composite.

28. The disposable diaper of claim 1 further comprising an acquisition layer and an impermeable layer, said core being disposed between said acquisition layer and said impermeable layer.

29. The disposable diaper of claim 1 further comprising a second nonwoven web and an acquisition layer, said acquisition layer being disposed between said core and said second nonwoven web.

30. The disposable diaper of claim 1, wherein said superabsorbent polymer comprises the reaction product of
a polymer derived from an α-β-ethylenically unsaturated carboxylic acid monomer, said polymer comprising neutralized carboxylic acid groups, and
a crosslinking agent.

31. The disposable diaper of claim 30, wherein said α-β-ethylenically unsaturated carboxylic acid is selected from the group consisting of methacrylic acid, crotonic acid, maleic acid, maleic acid anhydride, itaconic acid, fumaric acid, and mixtures thereof.

32. The disposable diaper of claim 30, wherein said polymer comprises polyacrylic acid.

33. The disposable diaper of claim 1, wherein said superabsorbent polymer remains disposed within the matrix of the high loft web when contacted with an aqueous composition.

34. The disposable diaper of claim 1, wherein said core further comprises cellulose fibers, said composite being disposed in regions on said cellulose fibers.

35. The disposable diaper of claim 1, wherein said core comprises a plurality of strips of said composite.

36. An absorbent article having a core that comprises a composite comprising:
superabsorbent polymer; and
a high loft nonwoven web impregnated with said superabsorbent polymer,
said superabsorbent polymer having been formed in situ,
said composite comprising from 10% by weight to about 90% by weight superabsorbent polymer.

37. An article according to the absorbent article of claim 36 selected from the group consisting of feminine napkins, incontinence pads and mattress pads.

38. An absorbent article having a core that comprises a composite comprising:
superabsorbent polymer; and
a nonwoven web impregnated with said superabsorbent polymer, said nonwoven web having loft and a density of no greater than 0.025 g/m³,
said superabsorbent polymer having been formed in situ,
said composite comprising from 10% by weight to about 90% by weight superabsorbent polymer.

39. The absorbent article of claim 38, wherein said nonwoven web has a density no greater than 0.023 g/m³.

40. A composite comprising:
superabsorbent polymer; and
a high loft nonwoven web impregnated with said superabsorbent polymer, said superabsorbent polymer having been formed in situ,
said composite comprising from 10% by weight to about 90% by weight superabsorbent polymer.

41. A method of making an absorbent article comprising
impregnating a high loft nonwoven web with an aqueous composition comprising a superabsorbent polymer precursor and a crosslinking agent;
drying said composition to form a composite comprising from 10% by weight to about 90% by weight superabsorbent polymer; and
incorporating said composite in an absorbent article.
